(19) United States
(12) Patent Application Publication
(10) Pub. No.: US 2012/0203235 A1
(43) Pub. Date: Aug. 9, 2012

(54) Device and Method for Bone Cement Removal

(76) Inventor: Richard Berger, Chicago, IL (US)

(21) Appl. No.: 13/368,608

(22) Filed: Feb. 8, 2012

Related U.S. Application Data
(60) Provisional application No. 61/440,653, filed on Feb. 8, 2011.

(51) Int. Cl. A61B 17/58

(52) U.S. Cl. 606/92

(57) ABSTRACT

A ribbon device and method of using the same can be employed to remove extravagated bone cement during an implantation procedure while minimizing or eliminating the use of mechanical force, heat, or potentially injurious chemical removal agents. The ribbon device bonds chemically or mechanically with substantially unset extravagated bone cement, resulting in removal of the extravagated bone cement when the device is used according to the methods of this invention. The ribbon device is formed of biocompatible mesh or weave materials tending to bond with bone cement chemically or mechanically, such as surgical grade fabrics, surgical tape, or mersilene tape. The ribbon device and method of its use can be used in conjunction with implants including tibial and femoral implants.

### CROSS-REFERENCES TO RELATED APPLICATIONS

This application claims priority to provisional application Ser. No. 61/440,653 filed on Feb. 8, 2011 by Dr. Richard Berger.

This application is related to copending U.S. Application “Device and Method for Bone Cement Removal” filed on the same day as the present application, by Dr. Richard Berger.

All of these applications are incorporated herein by reference. Statement Regarding Federally Sponsored Research or Development: Not applicable.

### BACKGROUND

It is common practice in orthopedic surgery to secure bone implants to bones in part through the use of bone cement. Several types of bone cement exist and are commonly used by surgeons, including polymethylmethacrylate (PMMA) based cements.

Surgeons performing implantation procedures routinely use bone cement to enhance the stability of the connection between a bone and bone implant. Surgeons typically apply bone cement at the anticipated interface of a bone surface prepared for implantation and a bone implant prior to positioning the implant in connection with the bone. For example, in the case of a tibial implant procedure, a surgeon may apply bone cement to the resected proximal tibia prior to positioning the tibial implant. For further example, in a femoral implant procedure, a surgeon may apply bone cement to the resected distal femur prior to positioning the femoral implant. A surgeon may apply bone cement to the implant instead of, or in addition to, the bone surface prepared for connection or interface with the implant. After the bone cement is applied, the implant can be positioned. As the implant is fitted into place, bone cement can be, and is often, squeezed or extravagated beyond the desired area of the interface of the bone and implant.

Bone cements, including PMMA, set rapidly, presenting a surgeon with compressed time frames in which to work once the cement is prepared for use. PMMA, for example, can substantially set in less than ten minutes, although complete curing typically requires several hours. Once the bone cement sets, the surgical procedure can be completed while curing of the bone cement continues. Once set, bone cement is a rigid material.

Because of the rapid setting and structural properties of bone cement, removal of extravagated bone cement after an implant has been positioned in connection with a bone surface presents a number of challenges to the surgeon. Attempting to remove extravagated bone cement prior to setting of the cement is difficult due to the compressed time frame in which the surgeon must work. It is also difficult to clean bone cement from the posterior aspects of a joint, as such aspects are typically hidden, as in, for example, an anterior approach total knee revision. Attempting to remove extravagated bone cement prior to setting of the cement also presents a risk of inadvertent alterations to the alignment, spacing, or attachment of the implant to the bone surface.

If removal of extravagated bone cement is attempted after the bone cement has substantially set, additional risks arise. To remove bone cement after it has substantially set, removal means such as mechanical force, chemical removal agents, or heat are often required. Such removal methods create a risk of injury to the joint and the surrounding tissue, and also introduce risks of alteration to the alignment, spacing, or attachment of the implant.

Failure to remove extravagated bone cement can result in discomfort to the patient, as well as reduced quality of outcomes from the implantation procedure.

Several methods for removing bone cement are known to the art. For example, Tarabichy, S. et al, U.S. Pat. No. 4,702,236, Oct. 27, 1987, describes several techniques for removing bone cement, including a method using a heated blade instrument for removing the bone cement. Huebsch, D. L., U.S. Pat. Nos. 4,873,969, Oct. 17, 1989 and 5,064,426, Nov. 12, 1991 describes use of a thermal chisel to remove bone cement. Kiester, D., U.S. Pat. No. 5,462,552 describes a heated wire tip instrument that is allowed to cool after embedded in the bone cement to facilitate removal. Schmidt et. al, U.S. Pat. No. 6,264,660 describes the application of chisels, pneumatic hammers, shockwaves, and other modes of manual force to remove bone cement. Vandewalle et. al., U.S. Pat. No. 6,190,392 describes the use of ultrasonic waves to remove bone cement.

The known methods for removal bone cement are generally optimized for the removal of all bone cement from a bone surface in connection with the removal or replacement of a previously placed implant. In this circumstance, the surgeon is unconcerned with the alignment, spacing, and attachment of the implant, since it is intended to be removed.

By contrast, when an implant is being placed, only extravagated bone cement is required to be removed and the implant is desired to be retained in its intended orientation. The methods and instruments known to the art have significant disadvantages for removing extravagated bone cement in this context, such as risks to injury to the bone or surrounding tissue, damage to the implant, or alterations to the alignment, spacing, or attachment of the implant.

It is a feature of this invention to provide a device and method for removing extravagated bone cement from the area of the interface of an implant and a bone without application of excessive manual force, chemical removal compounds, heat, or other potentially injurious agents, while minimizing the risk of altering the alignment, spacing, or attachment of the implant.

### SUMMARY

The present invention is directed to a device and method for removal of extravagated bone cement that satisfies the need for new or improved devices and methods for removing extravagated bone cement from the area of the interface between a bone and implant while minimizing the risks of injury to the patient and undesired alterations to the alignment, spacing, or attachment of the implant.

Versions of the invention are directed to a method for removing bone cement in an implantation procedure. In a method of versions of the invention, a ribbon is cut from material selected to bond with bone cement. Before the bone cement has finished substantially curing, the ribbon is placed in or adjacent to the area of anticipated interface between the bone prepared for implantation and an implant. Such placement can be accomplished by wrapping one or more ribbons wholly or partially around the perimeter of a bone or implant adjacent to the anticipated interface of the bone and implant. The ribbon is placed in or adjacent to the area of interface before the bone cement has finished substantially setting. The ribbon can be placed before the bone cement is applied, or, optionally, can be placed after the bone cement is applied, but before the implant is placed in connection with the bone prepared for implantation. When an implant is positioned in connection with a bone prepared for implantation, bone cement extravagates from the interface between the implant and the bone. Such extravagated bone cement contacts the ribbon and bonds to it, either chemically or mechanically. After the bone cement has substantially set, but before curing is complete, the ribbon is removed. Removal of the ribbon results in substantial removal of extravagated bone cement.

In one version of the invention, one or more ribbons are wrapped partially or wholly around the perimeter of an implant adjacent to the anticipated interface between the implant and a bone prepared for implantation according to the method described above. In another version of the invention, one or more ribbons are wrapped partially or wholly around the perimeter of a bone prepared for implantation adjacent to the anticipated interface of an implant and the prepared bone according to the method described above.

In another version of the invention, a ribbon is applied directly to extravagated bone cement in the area of the perimeter of the interface between an implant and a bone prepared for implantation after the implant has been positioned in connection with the bone, before the bone cement has substantially set. After the bone cement has substantially set, but before curing is complete, the ribbon is removed. Removal of the ribbon results in substantial removal of extravagated bone cement.

The versions of the method of this invention are suitable for use in tibial and femoral implants. In a version of this invention pertaining to a tibial implant, a ribbon is wholly wrapped around the perimeter of a tibial implant adjacent to the anticipated interface between the implant and the prepared proximal tibia. The ribbon can be wrapped before the bone cement is applied, or, optionally, can be wrapped after the bone cement is applied, but before the tibial implant is placed in connection with the prepared proximal tibia. The tibial implant is then positioned in connection with the prepared proximal tibia. Bone cement that extravagates from the interface between the tibial implant and the proximal tibia contacts the ribbon. After the bone cement has substantially set, but before it completely cures, the ribbon is removed, resulting in substantial removal of the extravagated bone cement.

In another version of this invention pertaining to a tibial implant, a ribbon is wrapped wholly around the perimeter of a proximal tibia prepared for implantation adjacent to the anticipated interface between a tibial implant and the prepared proximal tibia. The ribbon can be wrapped before the bone cement is applied, or, optionally, can be wrapped after the bone cement is applied, but before the tibial implant is placed in connection with the prepared proximal tibia. The tibial implant is positioned in connection with the prepared proximal tibia. Bone cement that extravagates from the interface between the tibial implant and the proximal tibia contacts the ribbon. After the bone cement has substantially set, but before it completely cures, the ribbon is removed, resulting in substantial removal of the extravagated bone cement.

In a version of this invention pertaining to a femoral implant, a ribbon is partially wrapped around the posterior portion of the perimeter of a femoral implant adjacent to the anticipated interface between the implant and the prepared posterior distal femur. The ribbon can be wrapped before the bone cement is applied, or, optionally, can be wrapped after the bone cement is applied, but before the femoral implant is placed in connection with the prepared distal femur. The femoral implant is positioned in connection with the prepared distal femur. Bone cement that extravagates from the interface between the implant and the distal femur contacts the ribbon. After the bone cement has substantially set, but before it completely cures, removal of the ribbon results in substantial removal of the extravagated bone cement.

The versions of this invention include a device for removing extravagated bone cement in an implantation procedure. The device of the versions of this invention includes a ribbon substantially comprised of material tending to bond with bone cement such that when the ribbon is placed in contact with extravagated bone cement before the bone cement has substantially set, the ribbon tends to form a bond with the extravagated bone cement as the bone cement substantially sets. Preferably, the ribbon of the versions of this invention tends to form a bond with extravagated bone cement of superior strength to the bond between the extravagated bone cement and a bone, implant, or non-extravagated bone cement. After a bond is formed between extravagated bone cement and the ribbon, removal of the ribbon results in removal of substantial amounts of extravagated bone cement.

A ribbon according to the versions of this invention is made of biocompatible material that tends to form a bone with bone cement, such as fabric, surgical tape, or a bone cement fiber weave. In one version of this invention, the ribbon is composed of material that tends to bond with bone cement chemically. In another version of this invention, the ribbon is composed substantially of fabric or similar material sufficiently porous to tend to bond with bone cement mechanically. In some versions of the invention, the ribbon is adhesive tape, such as surgical tape or mersilene tape. In some versions of the invention, the tendency of the ribbon to bond to bone cement is enhanced by impregnating the ribbon with bone cement material, preferably PMMA. In other versions of the invention, the ribbon is substantially composed of woven fibers of bone cement material, preferably PMMA.

In the Summary above and in the Description, and the claims below, and in the accompanying drawings, reference is made to particular features of the invention. It is to be understood that the disclosure of the invention in this specification includes all possible combinations of such particular features.

### BRIEF DESCRIPTION OF THE DRAWINGS

These and other features, aspects, and advantages of the present invention will become better understood with regard to the following description and accompanying drawings, where:

FIG. 1 shows a ribbon of various versions of the invention.

FIG. 2 shows a roll of ribbon material of various versions of the invention.

FIG. 3 shows a perspective view of the ribbon of various versions of this invention in use in connection with a tibial implant.

FIG. 4 shows a side view of the ribbon of various versions of this invention in use in connection with tibial implant and a proximal tibia.

FIG. 5 shows a side view of the ribbon of various versions of this invention in use in connection with a femoral implant.

FIG. 6 shows a side view of the ribbon of various versions of this invention in use in connection with femoral implant and a distal femur.

FIG. 7 shows a perspective view of removal of extravagated bone cement according to the various versions of this invention.

FIG. 8 shows a perspective view of a tibial implant and proximal tibia after extravagated bone has been removed according to the versions of this invention.

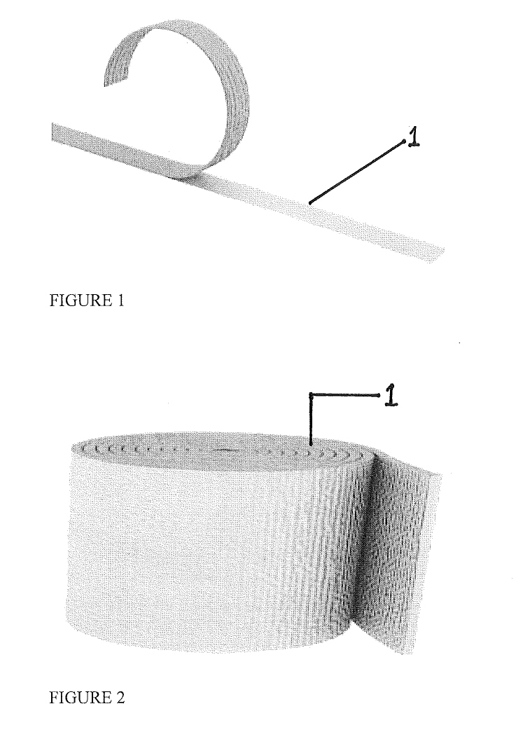

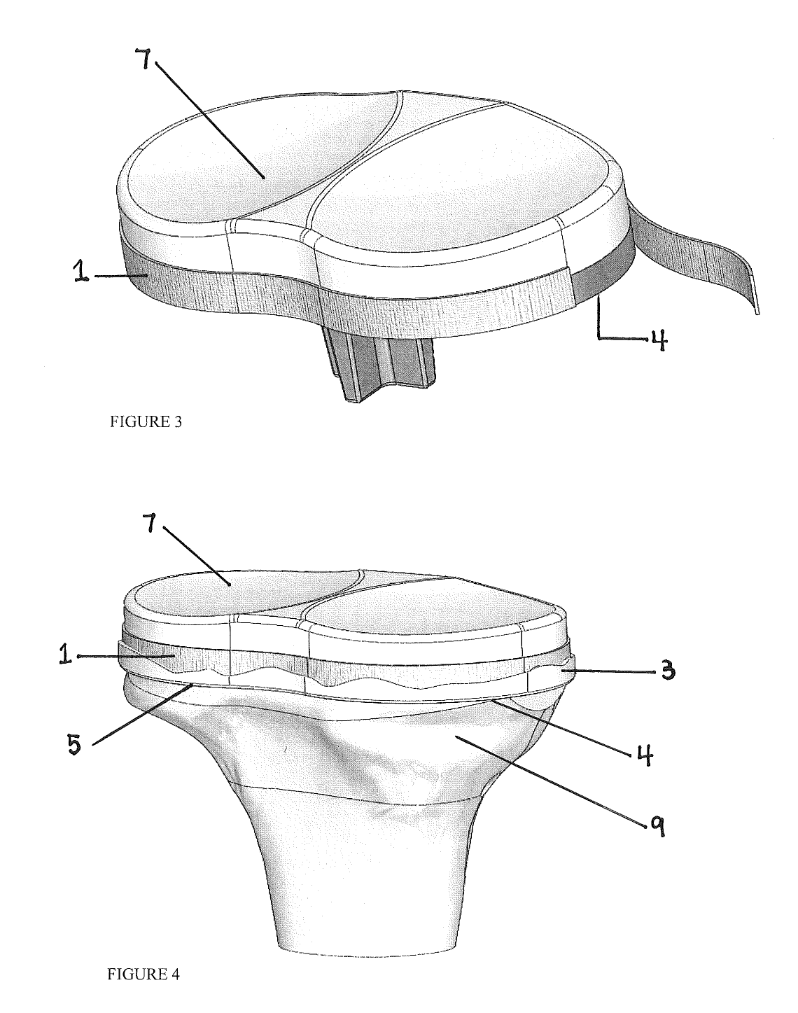

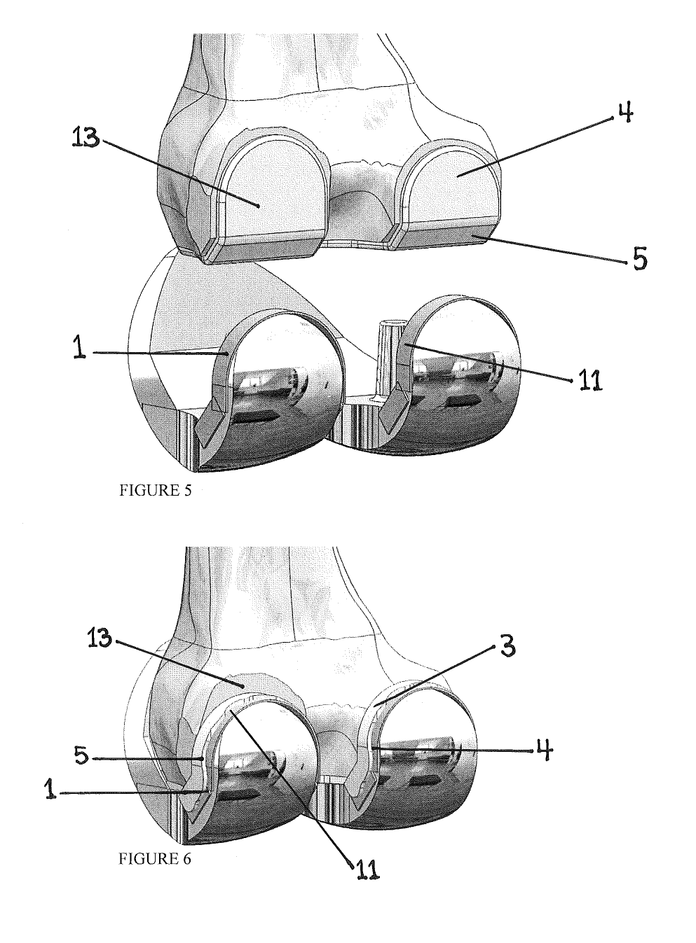

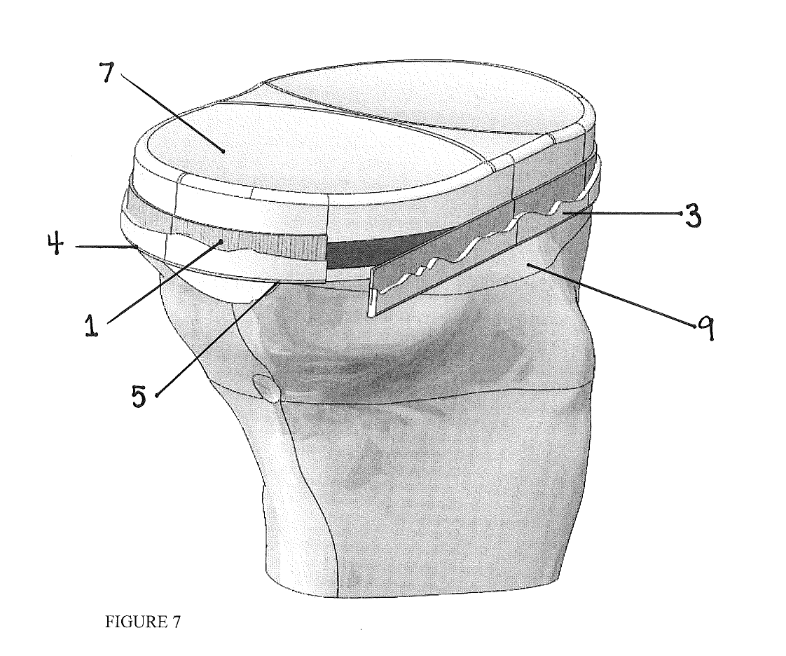

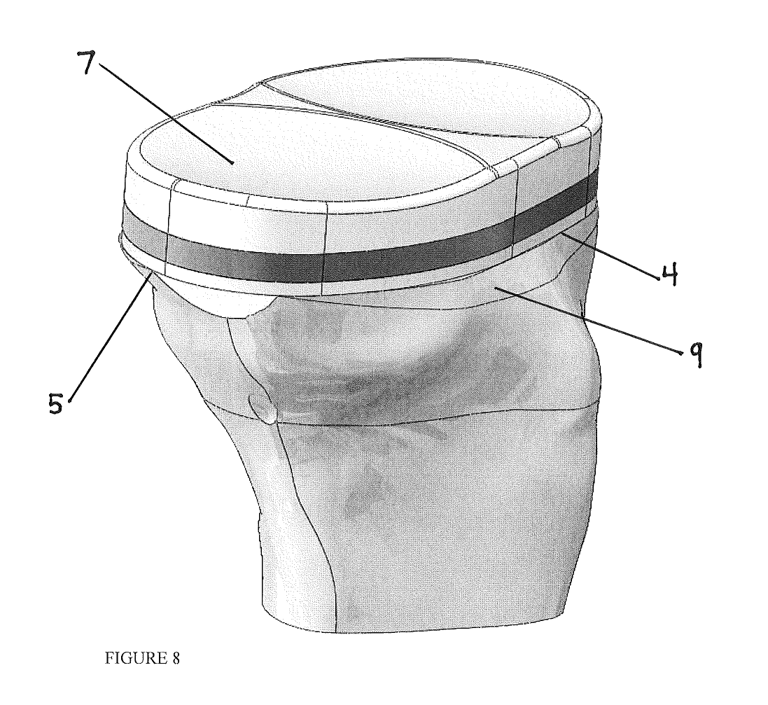

### DESCRIPTION

The versions of the present invention are directed towards a ribbon [1] device for use in removing extravagated bone cement [3] in an implantation procedure, and methods for using a ribbon [1] to remove of extravagated bone cement [3] in an implantation procedure.

A “ribbon” [1] according to the versions of the present invention is a flexible biocompatible material for use in removing extravagated bone cement [3] in an implantation procedure that forms a sufficient bone with bone cement such that if extravagated bone cement [3] contacts the ribbon prior to substantial setting, removal of the ribbon after the bone cement substantially sets, but before it substantially cures, will result in substantial removal of the extravagated bone cement [3].

A ribbon [1] according to the versions of the present invention can be composed of any biocompatible material or combination of biocompatible materials that is: (1) sufficiently flexible to permit the ribbon [1] to be placed in areas adjacent to the anticipated interface of a bone prepared for implantation and an implant, preferably to be wrapped wholly or partially around a bone or implant; and (2) tends to form a bond with extravagated bone cement [3] of superior strength to the bond formed between the extravagated bone cement [3] and a bone, implant, or non-extravagated bone cement.

A ribbon [1] according to the versions of this invention can be selected from any biocompatible material or materials that will tend to bond chemically or mechanically with bone cement, including materials such as cloth, fabric, fiber, and other weaves or meshes. The scope of the versions of this invention includes ribbons comprised of weaves or meshes woven or constructed from one or more biocompatible materials such as cloth, fabric, fiber, or metal. Preferably, a ribbon [1] according to versions of the present invention is formed of woven fiber material such as that used for surgical tape, particularly mersilene. Optionally, a ribbon [1] according to the versions of this invention can be an adhesive tape. A ribbon [1] according to the versions of this invention is optionally formed of a mesh or weave of any biocompatible material or materials that, as meshed or woven, form a structure sufficiently porous to bond mechanically to bone cement

Optionally, a ribbon [1] according to versions of this invention is a mesh or weave of biocompatible material or materials impregnated with an agent that enhances the bonding tendency of the ribbon [1]. Suitable agents include PMMA and other bone cements, and other compounds with similar properties that are readily recognized by those skilled in the art. A ribbon [1] according to versions of this invention is optionally substantially formed of such agents, and is optionally formed substantially of woven PMMA fibers.

A “bond” according to the versions of this invention is a mechanical or chemical bond formed when: (1) a ribbon [1] is placed into contact with extravagated bone cement [3] before the extravagated bone cement [3] has substantially set; and (2) the ribbon [1] remains substantially in contact with the extravagated bone cement [3] until the extravagated bone cement [3] has substantially set. A bond according to the versions of this invention is sufficiently strong such that, after the bond is formed, removal of the ribbon [1] results in substantial removal of extravagated bone cement [3] from any bone, implant, or non-extravagated bone cement material with which said extravagated bone cement is in contact.

A ribbon [1] according to the versions of this invention “tends” to form a bond with extravagated bone cement [3] if the ribbon forms a bond with the extravagated bone cement [3] superior to the bond between extravagated bone cement [3] and bone or non-extravagated bone cement when: (1) the ribbon [1] comes into contact with extravagated bone cement [3] before the extravagated bone cement [3] has substantially set; and (2) the ribbon [1] remains in substantial contact with the extravagated bone cement [3] until the extravagated bone cement [3] has substantially set or cured.

“Extravagated” or “excess” bone cement [3] according to the versions of this invention means bone cement that extravagates from the area of interface [4] of a bone prepared for implantation and an implant during the positioning the implant in connection with the bone prepared for implantation.

The “interface” [4] of an implant and a bone prepared for implantation means the surface or surfaces of contact between the bone prepared for implantation and the implant, to which bone cement [5] is applied. The “area of interface” is the area surrounding the interface into which bone cement could extravagate when the implant is placed or positioned in connection with the bone prepared for implantation. The “anticipated interface” is the surface of connection between the bone prepared for implantation and the implant intended by the medical provider performing the implantation procedure. It is within the scope of the versions of this invention for bone cement [5] to be applied to the bone prepared for implantation, the implant, or both.

An implant is “positioned in connection with” a bone prepared for implantation when it is fitted into its intended position, orientation, and alignment with the bone prepared for implantation after bone cement [5] has been applied.

“Bone cement” or “bone cement material” [5] includes any bone cement used in implantation procedures, such as PMMA or methylmethacrylate (MMA).

The devices and methods of versions of the present invention can be used with any bone cement appropriate for use in implantation procedures. Preferably, the devices and methods of versions of the present invention are used with PMMA bone cements. Optionally, the devices and methods of versions of this invention may be used with MMA bone cements, or other bone cements appropriate for use in implantation procedures. While the versions of the device and methods described in specific examples herein depict a tape ribbon [1] used with PMMA bone cement, other ribbon [1] types and other bone cements [5] are within the scope of the versions of this invention.

Methods of use of the versions of this invention involve placing a ribbon [1] adjacent to the anticipated interface [4] of the implant and the bone prepared for implantation, or in the area of interface, positioning the implant in connection with the bone prepared for implantation before bone cement [5] applied to at least some surfaces of the interface [4] has substantially set, then removing the ribbon [1] after the bone cement [5] has substantially set such that removal of the ribbon [1] results in substantial removal of extravagated bone cement [3]. Preferably, the ribbon [1] is placed by wrapping a ribbon wholly around the bone prepared for implantation, or wholly or partially around the implant, or both. Optionally, the ribbon [1] is wrapped around the interface [4] of the implant and bone prepared for implantation after the implant is positioned in connection with the bone prepared for implantation, but before the bone cement [5] has substantially set.

In a method of use of the versions of this invention in connection with a tibial implant [7], a ribbon [1] is wrapped around the perimeter of a tibial implant [7] adjacent to the anticipated interface [4] of the tibial implant [7] and a proximal tibia prepared for implantation [9]. Bone cement [5] is applied to some or all of the interface [4]. The tibial implant [7] is positioned in connection with the prepared proximal tibia [9]. Extravagated bone cement [3] from the interface [4] of the tibial implant [7] and the proximal tibia [9] contacts the ribbon [1]. After the extravagated bone cement [3] has substantially set, but before it completely cures, the ribbon [1] is removed, resulting in substantial removal of extravagated bone cement [3]. Optionally, the ribbon [1] is wrapped around the perimeter of the tibial implant [7] adjacent to the interface [4] of the tibial implant [7] and the prepared proximal tibia [9] after the bone cement [5] is applied, but before the tibial implant [7] is positioned in connection with the prepared proximal tibia [9].

In another version of the method of this invention pertaining to a tibial implant [7], a ribbon [1] is wrapped around a proximal tibia prepared for implantation [9] adjacent to the anticipated interface [4] of a tibial implant [7] and the prepared proximal tibia [9]. Bone cement [5] is applied to at least some of the interface [4]. The tibial implant [7] is positioned in connection with the prepared proximal tibia [9]. Extravagated bone cement [3] from the interface [4] of the tibial implant [7] and the prepared proximal tibia [9] contacts the ribbon [1]. After the extravagated bone cement [3] has substantially set, but before it completely cures, the ribbon [1] is removed, resulting in substantial removal of extravagated bone cement [3]. Optionally, the ribbon [1] is wrapped around the perimeter of the prepared proximal tibia [9] adjacent to the interface [4] of the tibial implant [7] and the prepared proximal tibia [9] after the bone cement [5] is applied, but before the tibial implant [7] is positioned in connection with the prepared proximal tibia [9].

In a version of the method of the present invention pertaining to a femoral implant, a ribbon [1] is partially wrapped around the posterior portion of a femoral implant [11] adjacent to the anticipated interface [4] of the femoral implant [11] and the prepared posterior distal femur [13]. Bone cement [5] is applied to the interface [4]. The femoral implant [11] is positioned in connection with the prepared distal femur [13]. Extravagated bone cement [3] from the interface [4] between the femoral implant [11] and the distal femur [13] contacts the ribbon [1]. After the extravagated bone cement [3] has substantially set, but before it completely cures, removal of the ribbon [1] results in substantial removal of the extravagated bone cement [3]. Optionally, the ribbon [1] can be applied to the femoral implant [11] after the bone cement [5] is applied, but before the femoral implant [11] is positioned in connection with the prepared distal femur [13].

What is claimed is:

1. A device for removing extravagated bone cement from the area of the interface of a bone and a bone implant, said device comprising a ribbon substantially formed of material that tends to bond with extravagated bone cement.

2. The device of claim 1, in which said ribbon tends to form a chemical bond with said extravagated bone cement.

3. The device of claim 1, in which said ribbon tends to form a mechanical bond with said extravagated bone cement.

4. The device of claim 1, in which said ribbon is comprised substantially of fabric.

5. The device of claim 1, in which said ribbon is comprised substantially of tape.

6. The device of claim 1, in which said ribbon is impregnated with bone cement prior to contact with said extravagated bone cement.

7. The device of claim 1, in which said ribbon is comprised substantially of bone cement fiber.

8. A method for removing extravagated bone cement from the area of the interface of a bone and an implant, said method comprising the steps of:
a. Placing a ribbon in the area of interface of a bone prepared for implantation and an implant, said ribbon substantially comprising material that tends to form a bond with extravagated bone cement;
b. Applying bone cement to the interface said bone and said implant;
c. Positioning said implant in connection with said bone prior to substantial setting of said bone cement;
d. Permitting said bone cement to substantially set; and
e. Removing said ribbon before said bone cement completely cures.

9. The method of claim 8, in which said ribbon is placed prior to positioning said implant in connection with said bone prepared for implantation.

10. The method of claim 8, in which said ribbon is placed after said implant is positioned in connection with said bone prepared for implantation.

11. The method of claim 8, in which said ribbon tends to form a chemical bond with said extravagated bone cement.

12. The method of claim 8, in which said ribbon tends to form a mechanical bond with said extravagated bone cement.

13. The method of claim 8, in which said ribbon is comprised substantially of fabric.

14. The method of claim 8, in which said ribbon is comprised substantially of tape.

15. The method of claim 8, in which said ribbon is impregnated with bone cement prior to contact with said extravagated bone cement.

16. The method of claim 8, in which said ribbon is comprised substantially of bone cement fiber.

17. The method of claim 8, in which said ribbon is placed in said area of interface by wrapping said ribbon around said bone prepared for implantation.

18. The method of claim 8, in which said ribbon is placed in said area of interface by wrapping said ribbon around said implant.

19. The method of claim 8, in which two or more ribbons are placed in said area of interface.

20. The method of claim 8, in which said implant is:
(a) a femoral implant; or
(b) a tibal implant.
Although the present invention has been described in considerable detail with reference to certain preferred versions thereof, other versions are possible. For example, materials, shapes, implant types, impregnating substances, bond types, or methods or method orders other than those described in detail herein may be used for the versions of this invention. Similarly, other steps may be included, or omitted from, the methods of the versions of this invention. Therefore, the spirit and scope of the claims should not be limited to the description of the preferred versions described herein.
